(19) United States
(12) Patent Application Publication
(10) Pub. No.: US 2025/0352544 A1
(43) Pub. Date: Nov. 20, 2025

(54) TREATMENT OF MUSCLE ATROPHY USING GSK-3 INHIBITORS

(71) Applicant: Technion Research & Development Foundation Limited, Haifa (IL)

(72) Inventors: Shenhav SHEMER, Haifa (IL); Dina AWEIDA, Haifa (IL)

(73) Assignee: Technion Research & Development Foundation Limited, Haifa (IL)

(21) Appl. No.: 19/213,051

(22) Filed: May 20, 2025

Related U.S. Application Data
(60) Provisional application No. 63/649,483, filed on May 20, 2024.

(51) Int. Cl. A61K 31/506; A61K 45/06; A61P 21/00

(52) U.S. Cl. CPC A61K 31/506; A61K 45/06; A61P 21/00

(57) ABSTRACT

Methods of treating muscle atrophy (e.g., reduced muscle mass or muscle loss) using GSK-3β inhibitors are provided.

### RELATED APPLICATION(S)

This application claims the benefit of priority of U.S. Provisional Patent Application No. 63/649,483 filed on May 20, 2024, the contents of which are incorporated herein by reference in their entirety.

### SEQUENCE LISTING STATEMENT

The XML file, entitled 103285SequenceListing.xml, created on May 20, 2025, comprising 4,127 bytes, submitted concurrently with the filing of this application is incorporated herein by reference.

### FIELD AND BACKGROUND OF THE INVENTION

The present invention, in some embodiments thereof, relates to therapy, and more particularly, but not exclusively, to compositions and methods usable in the treatment of subjects having, or being at risk of having, muscle atrophy.

There are approximately 600 skeletal muscles in the human body, accounting for about 45% of the total human body weight. Skeletal muscles play an important role in the body, including in thermoregulation, systemic metabolism, exercise, and visceral protection.

Muscle atrophy or wasting is a common condition characterized by the loss or decrease in muscle mass and strength, mitochondrial dysfunction, changes in muscle fiber type, and impaired physical function.

Muscle atrophy has a major impact on patients, by reducing the body's ability to respond to stress and chronic diseases, and thereby increasing morbidity and mortality and brings a huge socioeconomic burden and impacts patient prognosis. Muscle atrophy can impair limb movements, posing a potential risk to life as well as reduced quality of life for patients. The rapid loss of muscle mass and strength can lead to loss of muscle function, disability, frailty, reduced quality of life, and increased morbidity and mortality.

Muscle atrophy can be caused by an imbalance between rates of protein synthesis and degradation occurring as a result of factors such as aging, obesity, malnutrition (e.g., fasting), immobility, central nervous system damage, or can be associated with certain diseases, for example motor neuron diseases such as amyotrophic lateral sclerosis (ALS), cancer, congestive heart failure, chronic obstructive pulmonary disease, AIDS, liver disease, renal failure and cardiac failure.

The loss of muscle mass results largely from the accelerated degradation of the contractile myofibrils proteins, primarily by the ubiquitin-proteasome pathway. Their destruction accounts for the reduction in muscle strength and increased disability that reduce quality of life and contribute to mortality.

Aging-related muscle atrophy (sarcopenia) is a hallmark of aging, leading to weight loss, frailty, and reduced life expectancy, and preservation of muscle mass enhances health, quality of life, and longevity.

Loss of muscle mass and body weight in aging and cancer patients, known as cancer-associated cachexia, contribute to weakness, disability, frailty, and increased morbidity and mortality. Clinical studies indicate that cachexia is a major cause of death in cancer patients, and that preservation of muscle mass prolongs survival.

Currently, there are no effective FDA-approved therapies for muscle atrophy, and the only validated treatment is exercise, which reduces various types of atrophy and forms the mainstay of clinical management. However, exercise is not a practical option for bed-ridden, frail, sarcopenic or older individuals, or those with acute illnesses.

A number of treatments for muscle atrophy have been explored, which are primarily focused on anti-inflammatory and anti-oxidation actions, promoting protein synthesis, inhibiting protein degradation, and promoting muscle regeneration [Huang et al. Antioxidants (Basel). 2022 Dec 26;12(1):44]. Salvia miltiorrhiza, a Traditional Chinese Medicine containing magnesius lithospermate B, has also been used to prevent obesity-related skeletal muscle atrophy by inhibiting MAFbx and MuRF1-mediated muscle degeneration [Cheng et al., Nutrients. 2021;14:104]. The widely used drug for type 2 diabetes mellitus, Metformin, has been found to alleviate skeletal muscle atrophy in grx1 KO mice, reduce intramuscular lipid sediments, and increase glucose utilization through the AMPK/Sirt1 pathway [Yang et al., Biochem. Biophys. Res. Commun. 2020;533:1226-1232].

Glycogen synthase kinase-3 (GSK-3) is recognized as an important target for drug discovery and its inhibition has been considered a promising therapeutic approach for treating several pathologies including neurodegenerative diseases and malignancies. In humans, GSK-3 is expressed as two isozymes, GSK-3a and GSK-3β (SEQ ID NO:1) which are encoded by two genes and share high homology in their catalytic domains. The mechanisms by which GSK-3 is thought to contribute to pathogenesis are diverse. These include phosphorylation of the microtubule-associated protein tau, destabilization of the Wnt signaling component β-catenin, regulation of multiple transcription factors such as NF-κβ, activation of pro-inflammatory factors, and impairment of clearance pathways.

Some of the present inventors have previously uncovered that an initial key event that triggers the atrophy process involves the phosphorylation of the desmin cytoskeleton by glycogen synthase kinase-3 beta (GSK-3β; SEQ ID NO:1), rendering this enzyme a target of interest in the treatment of muscle atrophy [Aweida et al., J Cell Biol. 2018 Oct 1;217(10): 3698-3714]. It has been uncovered that phosphorylation of desmin filaments, which are critical for muscle architecture and function, by GSK-3β (SEQ ID NO:1), and a subsequent degradation, promote overall muscle protein breakdown and atrophy. It has been reported that GSK-3β (SEQ ID NO:1) inhibition in mice prevented desmin phosphorylation and depolymerization and blocked atrophy induced by fasting or denervation. Additionally, spaceflight, an activity known to cause muscle atrophy, was found to reduce GSK-3β (SEQ ID NO:1) content across all missions. It was further observed that inhibiting GSK-362 (SEQ ID NO:1) increases muscle mass, preserves muscle strength and promotes the oxidative fiber type with Earth-based hindlimb unloading [Baranowski et al., iScience. 2023 Jun 8;26(7):107047].

Bone marrow-derived miR-140 was found to inhibit endotoxic-induced glycolysis and atrophy of skeletal muscle by negatively regulating the WNT signaling pathway and simultaneously reducing the expression of Wnt family member 11, β-catenin, and GSK-3β (SEQ ID NO:1) [Liu et al., Am. J. Physiol. Cell Physiol. 2019;317:C189-C199].

GSK-3 inhibitors have been reviewed, for example, in Eldar-Finkelman et al., Front Mol Neurosci. 2011; 4: 32; and Arciniegas Ruiz et al., Front Mol Neurosci, 2022 Jan. 21;14:792364, and include inorganic substances, and organic small molecules and peptides.

Among the first synthetic small molecule GSK-3 inhibitors reported were pyrimidine or pyridine-based compounds, developed by Chiron (see, for example, PCT International Patent Application Publication No. WO 99/65897 and U.S. Patent Application Publication No. 2002/0156087). Compounds known as CHIR98014 (CT98014), CHIR98023 (CT98023), CHIR99021 (CT99021) were shown as highly potent and selective inhibitors of GSK-3. Compounds of the CHIR family were reported, amongst other activities, to enhance the levels of the survival motor neuron protein (SMN) in spinal muscular atrophy (Makhortova et al. (2011) Nat. Chem. Biol. 7, 544-552. See also, PCT International Patent Application Publication No. WO 2010/048273. U.S. Patent Application Publication No. 2009/0306045 describes the use of these GSK-3β inhibitors as an effective therapy for a number of autoimmune diseases.

WO 2004/052404 describes substrate-competitive GSK-3β inhibitors, designed based on the recognition motif of the enzyme, and accordingly featuring a short peptide sequence that terminates by a fatty acid residue. An exemplary such inhibitor is also known as L803-mts having the following amino acid sequence: Myr-Gly-Lys-Glu-Ala-Pro-Pro-Ala-Pro-Pro-Gln-{Ser(p)}-Pro-NH₂ (SEQ ID NO:2). See also Plotkin et al., (2003) J. Pharmacol. Exp. Ther. 305, 974-980; and Kaidanovich-Beilin et al., J. Pharmacol. Exp. Ther. 316, 17-24.

Additional Background Art includes Kramer et al., Int J Alzheimer Dis. 2012;2012:381029.

### SUMMARY OF THE INVENTION

According to an aspect of some embodiments of the present invention there is provided a method for treating muscle atrophy in a subject in need thereof, as described and defined herein in any of the respective embodiments and any combination thereof, the method comprising administering to the subject a therapeutically effective amount of a compound represented by Formula I:

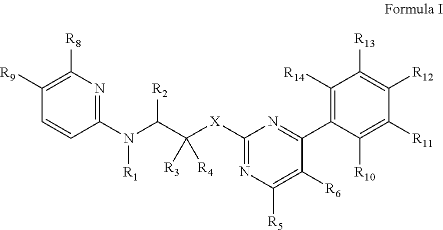

or a pharmaceutically acceptable salt thereof,
wherein:
X is O, NR₁₅, or CR₁₅R₁₆;
R₁, R₂, R₃ and R₄ are independently selected from hydrogen, hydroxy, thiol, alkyl, cycloalkyl, alkoxy, amine, aryl, alkaryl, heteroaryl, and heteroalicyclic;
R₅ is selected from hydrogen, halo, alkyl, cycloalkyl, alkoxy, thioalkoxy, amine, aryl, alkaryl, heteroaryl, heteroalicyclic, amide, thioamide and sulfonamide;
R₆ is selected from hydrogen, hydroxy, thiol, halo, carboxy, nitro, amine, amide, thioamide, cyano, alkyl, cycloalkyl, aryl, alkaryl, heteroaryl, heteroalicyclic, alkoxy, thioalkoxy, formyl, amide, sulfonyl, sulfonamide, and guanidinyl;
R₈ and R₉ are independently selected from hydrogen, nitro, amine, cyano, halo, thioamide, amide, oxime, guanidinyl, sulfonamide, carboxy, formyl, alkyl, cycloalkyl, aryl, alkaryl, heteroaryl and heteroalicyclic;
R₁₀, R₁₁, R₁₂, R₁₃ and R₁₄ are independently selected from hydrogen, nitro, amine, cyano, halo, thioamide, carboxy, hydroxy, thiol, amide, thioamide, alkyl, cycloalkyl, aryl, alkaryl, heteroaryl and heteroalicyclic; and
R₁₅ and R₁₆ are each independently selected from hydrogen, hydroxy, thiol, alkyl, cycloalkyl, alkoxy, amine, aryl, alkaryl, heteroaryl, and heteroalicyclic.

According to some embodiments of any of the embodiments described herein, X is NR₁₅.

According to some embodiments of any of the embodiments described herein, R₁₅ is hydrogen.

According to some embodiments of any of the embodiments described herein, at least one of R₈ and R₉ is selected from of nitro, amine, cyano, alkyl and alkoxy.

According to some embodiments of any of the embodiments described herein, at least one of R₈ and R₉ is selected from nitro, amine, alkyl and alkoxy.

According to some embodiments of any of the embodiments described herein, R₈ is an amine.

According to some embodiments of any of the embodiments described herein, R₉ is a nitro.

According to some embodiments of any of the embodiments described herein, each of R₁, R₂, R₃ and R₄ is hydrogen.

According to some embodiments of any of the embodiments described herein, R₅ is selected from hydrogen, aryl and heteroaryl.

According to some embodiments of any of the embodiments described herein, R₅ is hydrogen.

According to some embodiments of any of the embodiments described herein, at least one of R₁₀, R₁₁, R₁₂, R₁₃ and R₁₄ is other than hydrogen and is selected from halo, alkyl, hydroxy, alkoxy, amide and cyano.

According to some embodiments of any of the embodiments described herein, R₁₀, R₁₁ and R₁₃ are each hydrogen and at least one or both of R₁₂ and R₁₄ are each independently selected from halo, alkyl, hydroxy, alkoxy, amide and cyan.

According to some embodiments of any of the embodiments described herein, R₁₂ and R₁₄ are each independently halo (e.g., chloro).

According to some embodiments of any of the embodiments described herein, R₆ is a heteroaryl.

According to some embodiments of any of the embodiments described herein, R₆ is selected from pyridyl, pyrimidinyl, pyrrolindinyl, thiazolyl, indolyl, imidazolyl, oxadiazolyl, tetrazolyl, pyrazinyl, triazolyl, thienyl, furanyl, quinolinyl, pyrrolylpyridyl, benzothiazolyl, benzopyridyl, benzotriazolyl, and benzimidazolyl, each being optionally substituted.

According to some embodiments of any of the embodiments described herein, R₆ is an imidazole.

According to some embodiments of any of the embodiments described herein, the compound is represented by Formula III:

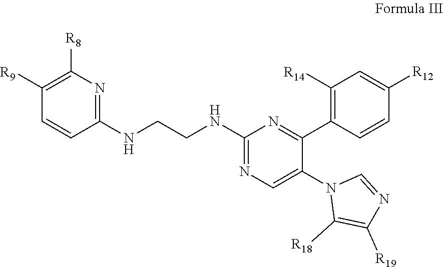

wherein:
R₁₂ and R₁₄ are each independently halo (e.g., chloro);
R₁₈ and R₁₉ are each independently selected from hydrogen, alkyl, cycloalkyl, aryl, and is preferably selected from hydrogen and alkyl; and
R₈ and R₉ are each independently selected from hydrogen, amine, cyano, nitro and alkyl.

According to some embodiments of any of the embodiments described herein, the compound is:

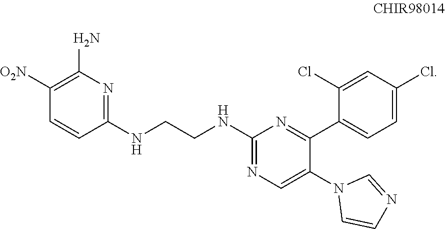 [SMILES: Nc1nc(NCCNc2ncc(-n3ccnc3)c(-c3ccc(Cl)cc3Cl)n2)ccc1[N+](=O)[O-]]

According to some embodiments of any of the embodiments described herein, the compound forms a part of a pharmaceutical composition which further comprises a pharmaceutically acceptable carrier.

According to some embodiments of any of the embodiments described herein, the muscle atrophy is associated with at least one of cachexia, sedentary lifestyle, sarcopenia, malnutrition, disuse atrophy, neurogenic atrophy, amyotrophic lateral sclerosis (ALS), Duchenne muscular dystrophy, myotonic dystrophy, Becker muscular dystrophy, facioscapulohumeral muscular dystrophy, limb-girdle muscular dystrophy, Charcot-Marie-Tooth disease, peripheral neuropathy, corticosteroid therapy, Emery-Dreifuss muscular dystrophy, Distal muscular dystrophy, Oculopharyngeal muscular dystrophy, Congenital muscular dystrophy, neuromuscular diseases, extended immobilization, trauma, alcoholism, cancer treatment, hyperthyroidism, heart failure, liver diseases, kidney diseases, diabetes, osteoarthritis, Cushing's syndrome, nutritional atrophy, severe burns, malabsorption syndromes, anorexia nervosa and ischemic atrophy, anorexia nervosa, rheumatoid arthritis and surgery.

According to some embodiments of any of the embodiments described herein, the method further comprises administering to the subject an additional therapeutically agent, the additional therapeutically active agent being selected from an agent usable in treating muscle atrophy, an additional GSK-3 inhibitor, and an agent that induces muscle atrophy, as described and defined in any of the respective embodiments and any combination thereof.

According to an aspect of some embodiments of the present invention there is provided a pharmaceutical composition comprising a GSK-3 inhibitor as described herein in any of the respective embodiments and any combination. The pharmaceutical composition can further comprises a pharmaceutically acceptable carrier, as described and defined herein. According to some embodiments of this aspect, the composition is identified for use in treating muscle atrophy, as described and defined herein in any of the respective embodiments. According to some embodiments of this aspect, the composition is identified for use in combination with one or more additional therapeutically active agent selected from an agent usable in treating muscle atrophy, an additional GSK-3 inhibitor, and an agent that induces muscle atrophy, as described and defined in any of the respective embodiments and any combination thereof. According to some embodiments of this aspect, the composition further comprises one or more additional therapeutically active agent selected from an agent usable in treating muscle atrophy, an additional GSK-3 inhibitor, and an agent that induces muscle atrophy, as described and defined in any of the respective embodiments and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph depicting the muscle weight of muscle isolated from fed mice expressing shLacz (Fed; control) and from 2-day fasting mice expressing shLacz or GSK-3β (SEQ ID NO: 1)-DNs (DN denotes dominant negative), as a percentage of the shLacz fed control.

FIG. 2 presents the chemical structures of exemplary GSK-3β inhibitors.

FIG. 3 is a bar graph depicting the mouse muscle weight (mg) in fed mice (control), fasting (atrophy-induced) mice, fasting (atrophy-induced) mice injected with CHIR98014 and fasting mice injected with L803-mts (N-Myristol-GKEAPPAPPQS(p)P; SEQ ID NO:2) (FIG. 2). **, p<0.005; p<0.0005 vs. Fed by ANOVA. ###, p<0.0005 vs. fasting by ANOVA.

FIGS. 4A-B present bar graphs showing the body weight (FIG. 4A) and the ratio of Tibialis Anterior (TA) muscle to body weight (BW) (FIG. 4B) in fed mice (control), fasting (atrophy-induced) mice, and fasting (atrophy-induced) mice treated with the GSK3-β inhibitor Tideglusib (20-30 mg/kg) via gavage. #, p<0.0001 vs. fed control by unpaired one tail Student t-test.

### DESCRIPTION OF SPECIFIC EMBODIMENTS OF THE INVENTION

The present invention, in some embodiments thereof, relates to therapy, and more particularly, but not exclusively, to compositions and methods usable in the treatment of subjects having, or being at risk of having, muscle atrophy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Based on previous findings that the initial key event that triggers the muscle atrophy process involves the phosphorylation of the desmin cytoskeleton by the protein kinase GSK-3β (SEQ ID NO: 1), as discussed in the Background section hereinabove, the present inventors have turned to test the ability of agents that reduce or inhibit GSK-3 activity and/or downregulate GSK-3 expression, particularly GSK-3β (SEQ ID NO:1), on the atrophy process. Such agents are also referred to herein collectively as GSK-3 inhibitors or GSK-3β inhibitors.

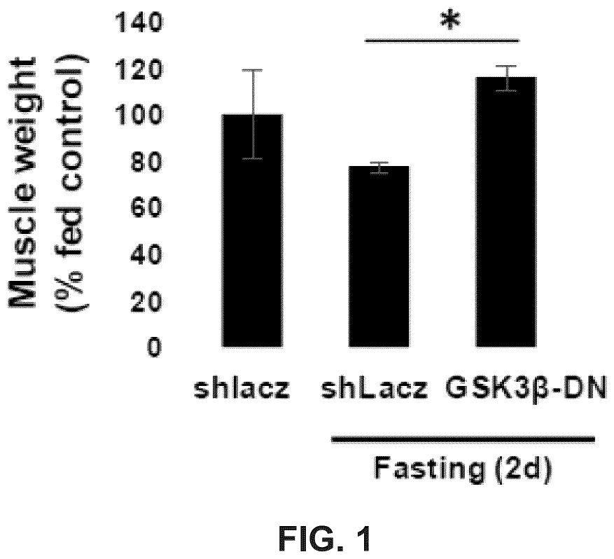

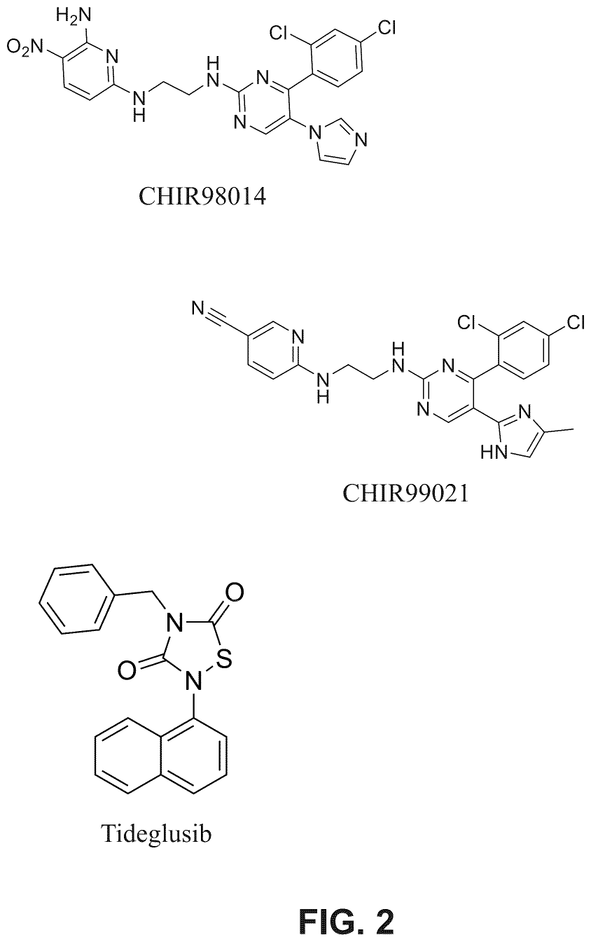

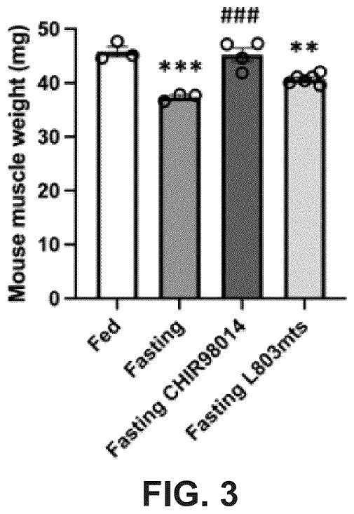

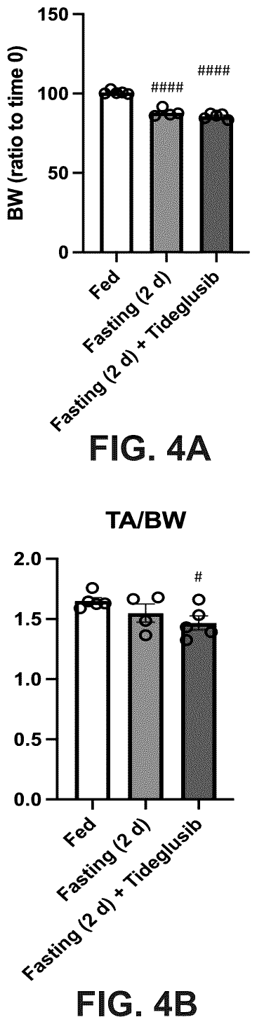

The present inventors have uncovered that CHIR98014 (see, FIG. 2), an exemplary compound that belongs to the family of pyrimidine or pyridine-based GSK-3β inhibitors disclosed in WO 99/65897, completely prevented the reduction in muscle mass in mice undergoing fasting-induced atrophy (as exemplified in Example 1 and FIG. 1). Another exemplary compound that belongs to the family of pyrimidine or pyridine-based GSK-3β inhibitors disclosed in WO 99/65897, CHIR99021 (see, FIG. 2), which feature some different structural features compared to CHIR98014, exhibited inferior activity. An exemplary peptide-based, substrate competitive GSK-3β inhibitor described in WO 2004/052404 (see, FIG. 2), L803-mts (SEQ ID NO:2) exhibited an inferior activity to that of CHIR98014 in preventing muscle loss under similar conditions (see, e.g., FIG. 3). The GSK-3β inhibitor Tideglusib (see, FIG. 2) was also tested and demonstrated minor effect in treating muscle atrophy (see, FIGS. 4A-B).

Embodiments of the present invention relate to compositions and methods employing GSK-3β inhibitors which are usable in the treatment or prevention of muscle atrophy.

According to an aspect of some embodiments of the present invention, there is provided a method of treating (eliminating, reducing or preventing progression of) muscle atrophy in a subject in need thereof. According to the present embodiments, the method is effected by administering the subject a therapeutically effective amount of a GSK-3 inhibitor (e.g., a GSK-3β inhibitor), thereby treating the muscle atrophy in the subject.

Exemplary GSK-3 inhibitors usable in the context of the present embodiments include, but are not limited to, those of the CHIR family, for example, as described in WO 99/65897 and U.S. Patent Application Publication No. 2002/0156087, including, for example, CHIR98014, CHIR98023, CHIR99021, or as collectively represented by Formula I, II or III herein, peptide inhibitors such as L803-mts, indirubin-3′-oxime, SB-216763, SB-415286, BIP-135, AZD1080, SAR502250, IMID1, IMID2, TWS119, AZD1080, SAR502250, JGK-263, AR-A014418, VP2.51, VP2.54, Kenpaullone, Alsterpaullone, Cazpaullone, Azakenpaullone, AZD2858, MMBO, TCS2002, PF-04802367, BRD0705, BRD3731, AF3581, TDZD-8, Tideglusib, VP0.7, VP3.35, SC100, L807mts, 5-imino-1,2,4-thiadiazoles, VP 1.14, and VP 1.16. Additional GSK-3 inhibitors are described, e.g., in PCT International Patent Application Publication Nos. WO 2001/049709, WO 2004/052404, WO 2012/101599, WO 2012/101601, WO 2014/207743, and WO 2022/044024.

According to some embodiments of any of the embodiments described herein, the GSK-3 inhibitor is not a substrate-competitive GSK-3 inhibitor.

According to some embodiments of any of the embodiments described herein, the GSK-3 inhibitor is a small molecule compound, and in some embodiments, it is not a peptide.

According to some embodiments of any of the embodiments described herein, the GSK-3 inhibitor is not a substrate-competitive peptide GSK-3 inhibitor.

The phrase “substrate-competitive” in the context of GSK-3 inhibitors describes, compounds (e.g., peptides or small molecules) that bind to the catalytic domain of the GSK-3, to which its natural substrate binds, and as such inhibit its catalytic activity by blocking this site and preventing the binding of the substrate thereto. Non-limiting examples of such substrate-competitive GSK-3 inhibitors are described, for example, in PCT International Patent Application Publication Nos. WO 2001/049709, WO 2004/052404, WO 2012/101599, WO 2012/101601, WO 2014/207743, and WO 2022/044024.

According to some embodiments of any of the embodiments described herein, the GSK-3 inhibitor is an ATP-competitive GSK-3 inhibitor, preferably a selective ATP-competitive GSK-3 inhibitor.

The phrase “ATP-competitive” in the context of GSK-3 inhibitors describes, As used herein, describes a compound (e.g., small molecule), or agent that inhibits the enzymatic activity of glycogen synthase kinase-3 (GSK-3), including GSK-3a and/or GSK-3β isoforms, by directly binding to the ATP-binding site of the enzyme's catalytic domain, thereby preventing or reducing the phosphorylation of GSK-3 substrates. Such inhibition occurs through competitive interaction with ATP, the natural substrate of the kinase, such that the inhibitor and ATP vie for the same binding pocket on GSK-3.

ATP-competitive inhibitors can be reversible or irreversible inhibitors, both are encompassed herewith.

According to some embodiments of any of the embodiments described herein, non-limiting examples of GSK-3 inhibitors include, but are not limited to, those of the CHIR family, for example, CHIR98014, CHIR98023, CHIR99021, or as collectively represented by Formula I, II or III herein, indirubin-3′-oxime, SB-216763, SB-415286, BIP-135, AZD1080, SAR502250, IMID1, IMID2, TWS119, AZD1080, SAR502250, JGK-263, AR-A014418, VP2.51, VP2.54, Kenpaullone, Alsterpaullone, Cazpaullone, Azakenpaullone, AZD2858, MMBO, TCS2002, PF-04802367, BRD0705, BRD3731, AF3581, TDZD-8, Tideglusib, VP0.7, VP3.35, SC100, 5-imino-1,2,4-thiadiazoles, VP 1.14, and VP 1.16.

According to some embodiments of any of the embodiments described herein, the GSK-3 inhibitor is a small molecule inhibitor, for example, a GSK-3 inhibitor of the CHIR family, such as disclosed in WO 99/65897.

According to some embodiments of any of the embodiments described herein, GSK-3 inhibitors usable in the method as described herein can be collectively represented by Formula I:

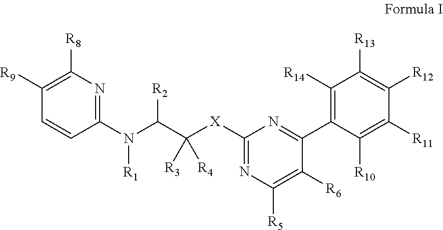

wherein:
X is O, NR₁₅, or CR₁₅R₁₆;
R₁, R₂, R₃ and R₄ are independently selected from hydrogen, hydroxy, thiol, alkyl, cycloalkyl, alkoxy, amine, aryl, alkaryl, heteroaryl, and heteroalicyclic;
R₅ is selected from hydrogen, halo, alkyl, cycloalkyl, alkoxy, thioalkoxy, amine, aryl, alkaryl, heteroaryl, heteroalicyclic, amide, thioamide and sulfonamide;
R₆ is selected from hydrogen, hydroxy, thiol, halo, carboxy, nitro, amine, amide, thioamide, cyano, alkyl, cycloalkyl, aryl, alkaryl, heteroaryl, heteroalicyclic, alkoxy, thioalkoxy, formyl, amide, sulfonyl, sulfonamide, and guanidinyl;
R₈ and R₉ are independently selected from hydrogen, nitro, amine, cyano, halo, thioamide, amide, oxime, guanidinyl, sulfonamide, carboxy, formyl, alkyl, cycloalkyl, aryl, alkaryl, heteroaryl and heteroalicyclic;
R₁₀, R₁₁, R₁₂, R₁₃ and R₁₄ are independently selected from hydrogen, nitro, amine, cyano, halo, thioamide, carboxy, hydroxy, thiol, amide, thioamide, alkyl, cycloalkyl, aryl, alkaryl, heteroaryl and heteroalicyclic; and
R₁₅ and R₁₆ are each independently selected from hydrogen, hydroxy, thiol, alkyl, cycloalkyl, alkoxy, amine, aryl, alkaryl, heteroaryl, and heteroalicyclic, or pharmaceutically acceptable salts thereof.

According to some embodiments of any of the embodiments described herein for Formula I, X is NR₁₅.

According to some embodiments of any of the embodiments described herein, R₁₅ is hydrogen.

According to some embodiments of any of these embodiments, X is NR₁₅ and R₁₅ is selected from hydrogen, alkyl, cycloalkyl, aryl, alkaryl, heteroaryl, and heteroalicyclic.

According to some embodiments of any of these embodiments, X is NR₁₅ and R₁₅ is hydrogen.

According to some embodiments of any of the embodiments described herein, at least one of R₈ and R₉ is selected from nitro, amine, cyano, alkyl and alkoxy.

According to some of any of these embodiments, R₈ and R₉ are each independently selected from amine, cyano, nitro and alkyl.

According to some embodiments of any of the embodiments described herein, at least one or each of R₈ and R₉ is independently selected from nitro, amine, alkyl and alkoxy.

According to some embodiments of any of the embodiments described herein, at least one of R₈ and R₉ is nitro or amine.

According to some embodiments of any of the embodiments described herein, one of R₈ and R₉ is nitro and the other one of R₈ and R₉ is amine.

According to some embodiments of any of the embodiments described herein, one of R₈ and R₉ is nitro and the other one of R₈ and R₉ is amine.

According to some embodiments of any of the embodiments described herein, R₈ is amine, preferably primary amine as described herein and R₉ is nitro.

According to some embodiments of any of these embodiments, at least one of R₈ and R₉ is selected from nitro, amine, cyano, alkyl and alkoxy, X is NR₁₅ and R₁₅ is selected from hydrogen, alkyl, cycloalkyl, aryl, alkaryl, heteroaryl, and heteroalicyclic.

According to some embodiments of any of these embodiments, at least one of Rg and R₉ is selected from nitro, amine, cyano, alkyl and alkoxy, X is NR₁₅ and R₁₅ is hydrogen.

According to some embodiments of any of these embodiments, at least one of R₈ and R₉ is selected from nitro, amine, alkyl and alkoxy, X is NR₁₅ and R₁₅ is hydrogen.

According to some embodiments of any of these embodiments, at least one of R₈ and R₉ is nitro, and the other one is amine, as described herein, X is NR₁₅ and R₁₅ is hydrogen.

According to some embodiments of any of the embodiments described herein, at least one, or at least two, or at least three, or each of R₁, R₂, R₃ and R₄ is hydrogen.

According to some embodiments of any of the embodiments described herein, each of R₁, R₂, R₃ and R₄ is hydrogen.

According to some embodiments of any of these embodiments, at least one of R₈ and R₉ is nitro, and the other one is amine, as described herein, and each of R₁, R₂, R₃ and R₄ is hydrogen. According to some of these embodiments, X is NR₁₅ and R₁₅ is hydrogen.

According to some embodiments of any of the embodiments described herein, at least one of R₁₀, R₁₁, R₁₂, R₁₃ and R₁₄ is selected from halo, alkyl, hydroxy, alkoxy, amide and cyano.

According to some embodiments of any of the embodiments described herein, at least one or at least two of R₁₀, R₁₁, R₁₂, R₁₃ and R₁₄ is/are independently halo (e.g., chloro).

According to some embodiments of any of the embodiments described herein, R₁₀, R₁₁ and R₁₃ are hydrogen and R₁₂ and R₁₄ are each independently selected from halo, alkyl, hydroxy, alkoxy, amide and cyano.

According to some of any of these embodiments, at least one of R₁₂ and R₁₄ is halo (e.g., chloro). According to some embodiments of any of the embodiments described herein, R₁₂ and R₁₄ are each independently halo (e.g., chloro). According to some embodiments of any of the embodiments described herein, R₁₂ and R₁₄ are each chloro.

According to some embodiments of any of the embodiments described herein, R₁₀, R₁₁ and R₁₃ are hydrogen and at least one or each of R₁₂ and R₁₄ is halo (e.g., chloro).

According to some embodiments of any of the embodiments described herein, R₁₀, R₁₁ and R₁₃ are hydrogen and R₁₂ and R₁₄ are each independently halo (e.g., chloro).

According to some embodiments of any of the embodiments described herein, R₈ and R₉ are each independently other than hydrogen and selected from nitro, alkyl, alkoxy, amine and cyano; X is NR₁₅ and R₁₅ is hydrogen; each of R₁, R₂, R₃ and R₄ is hydrogen; R₁₀, R₁₁ and R₁₃ are hydrogen and R₁₂ and R₁₄ are each independently selected from halo, alkyl, hydroxy, alkoxy, amide and cyano. According to some of these embodiments, R₁₂ and R₁₄ are each independently halo, and in some embodiments, R₁₂ and R₁₄ are each chloro.

According to some embodiments of any of the embodiments described herein, at least one of R₅ and R₆ is other than hydrogen.

According to some embodiments of any of the embodiments described herein, at least one or each of R₅ and R₆ is independently an aryl or heteroaryl, each can be substituted or unsubstituted.

According to some embodiments of any of the embodiments described herein, R₅ is selected from hydrogen, aryl and heteroaryl and is preferably hydrogen. According to some embodiments of any of the embodiments described herein, R₅ is hydrogen and R₆ is aryl or heteroaryl as described herein.

According to some embodiments of any of the embodiments described herein, R₆ is a heteroaryl as defined herein. According to some of these embodiments, R₅ is hydrogen.

According to some embodiments of any of the embodiments described herein, R₆ is selected from pyridyl, pyrimidinyl, pyrrolindinyl, thiazolyl, indolyl, imidazolyl, oxadiazolyl, tetrazolyl, pyrazinyl, triazolyl, thienyl, furanyl, quinolinyl, pyrrolylpyridyl, benzothiazolyl, benzopyridyl, benzotriazolyl, and benzimidazolyl, each being unsubstituted or substituted, as defined herein.

According to some of any of these embodiments, R₆ selected from an imidazolyl, a triazolyl, and a pyridyl, each being optionally substituted.

According to some embodiments of any of the embodiments described herein, R₅ is hydrogen R₆ is an imidazole.

According to some embodiments of any of the embodiments described herein, R₆ is an imidazole.

According to some embodiments of any of the embodiments described herein, R₆ is an unsubstituted imidazole.

According to some embodiments, R₆ is a substituted imidazolyl. In some such embodiments, R₆ is an imidazolyl substituted by one or more of alkyl, cycloalkyl, aryl. In some embodiments, R₆ is an imidazolyl substituted by one or more alkyls, preferably lower alkyls (e.g., methyl).

According to some of any of these embodiments, the GSK-3 inhibitor is represented by Formula III:

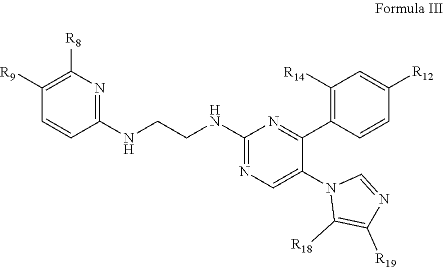

wherein:
R₁₂ and R₁₄ are each independently selected from halo, alkyl, hydroxy, alkoxy, amide and cyano;
R₁₈ and R₁₉ are each independently selected from hydrogen, alkyl, cycloalkyl, aryl; and
R₈ and R₉ are each independently selected from hydrogen, amine, cyano, nitro and alkyl.

According to some of any of these embodiments relating to Formula III, one or each of R₁₂ and R₁₄ is independently halo and in some embodiments, each of R₁₂ and R₁₄ is chloro.

According to some of any of these embodiments relating to Formula III, R₈ and R₉ are as described herein in any of the respective embodiments relating to Formula I.

According to some of any of these embodiments relating to Formula III, at least one of R₈ and R₉ is other than hydrogen, and in some embodiments, at least one or each of R₈ and R₉ is nitro or amine, as described herein in any of the respective embodiments relating to Formula I.

According to some of any of these embodiments relating to Formula III, R₈ is an amine, preferably a primary amine.

According to some of any of these embodiments relating to Formula III, R₉ is a nitro. According to some of any of these embodiments relating to Formula III, R₉ is a nitro and R₈ is an amine.

According to some of any of these embodiments relating to Formula III, at least one, or both, of R₉ and R₈ is not cyano.

According to some of any of these embodiments relating to Formula III, R₁₈ and R₁₉ are each independently hydrogen, such that the imidazole is an unsubstituted imidazole. In some embodiments, one or each of R₁₈ and R₁₉ is other than hydrogen, and is selected from alkyl, cycloalkyl, aryl. In some embodiments, one or each of R₁₈ and R₁₉ is alkyl, preferably a lower alkyl such as methyl.

According to some of any of these embodiments relating to Formula III, R₈ is an amine and one or both of R₁₂ and R₁₄ are independently chloro.

According to some of any of these embodiments relating to Formula III, R₉ is a nitro and one or both of R₁₂ and R₁₄ are independently chloro.

According to some of any of these embodiments relating to Formula III, R₉ is a nitro, R₈ is an amine, and one or both of R₁₂ and R₁₄ are independently chloro.

According to exemplary embodiments, the compound is:

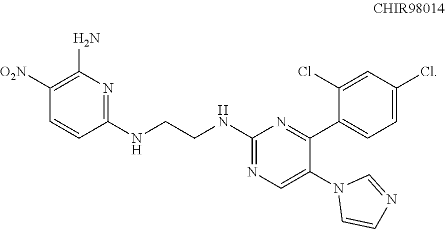 [SMILES: Nc1nc(NCCNc2ncc(-n3ccnc3)c(-c3ccc(Cl)cc3Cl)n2)ccc1[N+](=O)[O-]]

According to some embodiments of any of the embodiments described herein, the compounds as described herein are collectively represented by Formula II:

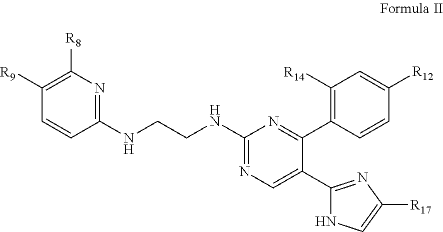

wherein:
R₁₂ and R₁₄ are each independently each independently selected from halo, alkyl, hydroxy, alkoxy, amide and cyano;
R₁₇ is selected from hydrogen, alkyl, cycloalkyl, aryl, and is preferably selected from hydrogen and alkyl (e.g., methyl); and
R₈ and R₉ are each independently selected from hydrogen, amine, cyano, nitro and alkyl.

According to some of any of the embodiments described herein for Formula II, one or each of R₁₂ and R₁₄ is independently halo, and in some embodiments, each of R₁₂ and R₁₄ is independent halo, e.g.., chloro.

According to some of any of the embodiments described herein for Formula II, R₈ is hydrogen.

According to some of any of the embodiments described herein for Formula II, one or both of R₈ and R₉ is other than hydrogen.

According to some of any of the embodiments described herein for Formula II, R₈ is hydrogen and R₉ is other than hydrogen.

According to some of any of these embodiments described herein for Formula II, R₉ is a cyano.

According to some of any of these embodiments described herein for Formula II, R₉ is a cyano and R₈ is hydrogen.

According to some of any of these embodiments described herein for Formula II, R₁₇ is an alkyl, preferably a lower alkyl such as methyl.

According to some of any of these embodiments described herein for Formula II, R₁₇ is an alkyl and R₈ is hydrogen.

According to some of any of these embodiments described herein for Formula II, R₁₇ is an alkyl and R₉ is a cyano.

According to some of any of these embodiments described herein for Formula II, R₁₇ is an alkyl, R₉ is a cyano and R₈ is hydrogen.

According to some of any of these embodiments described herein for Formula II, R₁₇is an alkyl, R₉ is a cyano and R₈ is hydrogen, and one or both of R₁₂ and R₁₄ are independently chloro.

According to an exemplary embodiments, the compound is:

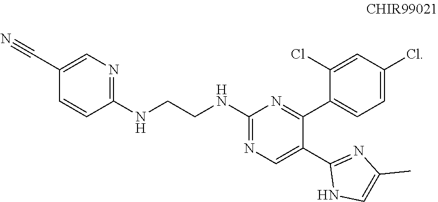 [SMILES: Cc1c[nH]c(-c2cnc(NCCNc3ccc(C#N)cn3)nc2-c2ccc(Cl)cc2Cl)n1]

According to some of any of the embodiments described herein, the GSK-3 inhibitor is not encompassed by Formula II as described herein in any of the respective embodiments.

According to some of any of the embodiments described herein, the GSK-3 inhibitor is encompassed by Formula I as described herein but is not encompassed by Formula II as described herein in any of the respective embodiments.

It is to be noted that compounds which are encompassed by Formula II or Formula III are also encompassed by Formula I. Compounds of Formula II and compounds of Formula III generally differ in the connectivity of their R₆ (imidazolyl) group.

According to some of any of the embodiments described herein, the GSK-3 inhibitor is not CHIR99021.

According to some of any of the embodiments described herein, the GSK-3 inhibitor is collectively represented by Formula IV:

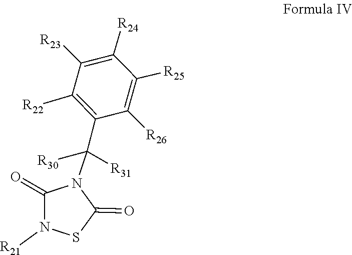

wherein:
R₂₁ is an aryl, alkaryl or is collectively referred to as an aryl-containing substituent,
R₃₀, R₃₁, R₂₂, R₂₃, R₂₄, R₂₅, and R₂₆ are each independently selected from hydrogen, alkyl, cycloalkyl, alkenyl, aryl, heteroaryl, heteroalicyclic, —COR₂₇, —C(O)OR₂₇, —C(O)NR₂₇R₂₈, —C═NR₂₇, —CN, —OR₂₇, —OC(O)R₂₇, —S(O)ₜ—R₂₇, —NR₂₇R₂₈, —NR₂₇C(O)R₂₈, —NO₂, —N═CR₂₇R₂₈ and halo,
t is selected from 0, 1, 2 and 3,
R₂₇ and R₂₈ are each independently selected from hydrogen, alkyl, cycloalkyl, alkenyl, aryl, heteroaryl, heteroalicyclic, alkoxy, aryloxy, and halo;
or a pharmaceutically acceptable salt thereof.

As used herein, the phrase “aryl-containing substituent” describes a substituent group that contains at least one aryl (as defined herein), optionally substituted and/or fused, including an aryl per se, or an alkaryl, as defined herein.

Non-limiting examples include

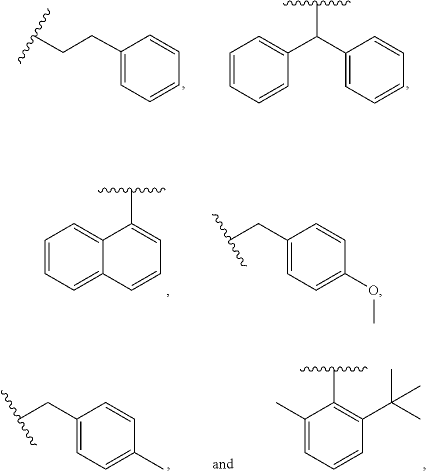

wherein the wavy lines indicate the connection of the substituent to the core structure.

According to some of any of these embodiments relating to Formula IV, R₂₁ is

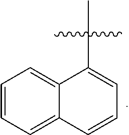 [SMILES: *c1cccc2ccccc12]

According to some of any of these embodiments relating to Formula IV, at least one or each of R₃₀ and R₃₁ is hydrogen.

According to some of any of these embodiments relating to Formula IV, at least one, or at least two, or at least three, or at least four, or all, of R₂₂, R₂₃, R₂₄, R₂₅, and R₂₆, is each hydrogen.

According to some of any of these embodiments relating to Formula IV, R₂₁ is

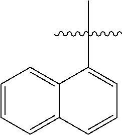 [SMILES: *c1cccc2ccccc12]

and at least one or each of R₃₀ and R₃₁ is hydrogen.

According to some of any of these embodiments relating to Formula IV, R₂₁ is

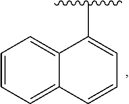 [SMILES: *c1cccc2ccccc12]

sand at least one, or at least two, or at least three, or at least four, or all, of R₂₂, R₂₃, R₂₄, R₂₅, and R₂₆, is each hydrogen.

According to some of any of these embodiments relating to Formula IV, at least one or each of R₃₀ and R₃₁ is hydrogen, and at least one, or at least two, or at least three, or at least four, or all, of R₂₂, R₂₃, R₂₄, R₂₅, and R₂₆, is each hydrogen.

According to an exemplary embodiments, the compound is Tideglusib (see, FIG. 2). According to some of any of the embodiments described herein, the GSK-3 inhibitor is not encompassed by Formula IV as described herein in any of the respective embodiments.

According to some of any of the embodiments described herein, the GSK-3 inhibitor is not Tideglusib.

It is to be noted that in all Formulae I, II, III and IV, any position at which no substituent is explicitly depicted is understood to bear one or more hydrogen atoms as substituents, in accordance with the valency requirements of the atom at that position. According to some embodiments of any of the embodiments described herein, a method as described herein is for treating or preventing muscle atrophy in a subject need thereof.

Herein, treating or preventing muscle atrophy encompasses treatment or preventing muscle loss or reduced muscle mass and/or strength.

According to some embodiments of any of the embodiments described herein, a method as described herein is for treating or preventing muscle loss or reduced muscle mass in a subject in need thereof.

According to some embodiments of any of the embodiments described herein, a method as described herein is for increasing muscle mass and/or strength or for preventing a reduction in muscle mass and/or strength in a subject in need thereof.

Subject to be treated according to the present embodiments include subjects identified as exhibiting or as being at risk of developing one or more symptoms and/or markers (e.g., biomarkers) of muscle atrophy, and/or (diagnosed as) afflicted by a disease or disorder that is known to cause muscle atrophy or that is treated by medications that are known to cause muscle atrophy.

Non-limiting examples of symptoms and/or markers of muscle atrophy include reduced muscle mass, decreased muscle strength, diminished physical endurance, impaired mobility, elevated circulating myostatin levels, elevated expression of muscle atrophy biomarkers (e.g., Atrogin-1, MuRF1) (compared to healthy subjects), and increased SMAD2/3 phosphorylation (compared to healthy subjects).

Non-limiting examples of conditions, diseases or disorders that are known to cause muscle atrophy or that are treated by medications that are known to cause muscle atrophy include cachexia, spaceflight, sedentary lifestyle, sarcopenia, malnutrition, disuse atrophy, neurogenic atrophy, amyotrophic lateral sclerosis (ALS), Duchenne muscular dystrophy, myotonic dystrophy, Becker muscular dystrophy, facioscapulohumeral muscular dystrophy, limb-girdle muscular dystrophy, Charcot-Marie-Tooth disease, peripheral neuropathy, corticosteroid therapy, Emery-Dreifuss muscular dystrophy, Distal muscular dystrophy, Oculopharyngeal muscular dystrophy, Congenital muscular dystrophy, neuromuscular diseases, extended immobilization, trauma, alcoholism, cancer treatment, hyperthyroidism, heart failure, liver diseases, kidney diseases, diabetes, osteoarthritis, Cushing's syndrome, nutritional atrophy, severe burns, malabsorption syndromes, anorexia nervosa and ischemic atrophy, rheumatoid arthritis, spaceflight and surgery.

According to some embodiments of any of the embodiments described herein, the muscle atrophy is associated with spaceflight, such that the subject participates or participated in a spaceflight and exhibits muscle atrophy or is prone to exhibit muscle atrophy. According to some of these embodiments, a method as described herein is for increasing muscle mass and/or strength or for preventing a reduction in muscle mass and/or strength in the subject.

According to some embodiments of any of the embodiments described herein, the muscle atrophy is associated with cachexia.

According to some embodiments of any of the embodiments described herein, the cachexia is associated with cancer, congestive heart failure, chronic obstructive pulmonary disease (COPD), chronic kidney disease and Acquired Immune Deficiency Syndrome (AIDS), burns, trauma, sarcopenia, denervation, disuse, and fasting. According to some of these embodiments, the subject is afflicted by cancer, congestive heart failure, chronic obstructive pulmonary disease (COPD), chronic kidney disease, Acquired Immune Deficiency Syndrome (AIDS), burns, trauma, sarcopenia, denervation, disuse, or is a fasting subject, and exhibits cachexia. According to some of these embodiments, the subject is afflicted with cancer, and is treated with anti-cancer therapy (e.g., chemotherapy, radiation) that can lead to cachexia or muscle loss.

The phrase “sedentary lifestyle”, as known in the art, describes a behavioral pattern characterized by low levels of physical activity and prolonged periods of inactivity, which may lead to disuse-related muscle atrophy. According to some of these embodiments, the subject exhibits such a behavioral pattern.

The term “malnutrition”, as known in the art, describes a condition resulting from inadequate intake or absorption of nutrients necessary for health and muscle maintenance. According to some of these embodiments, the subject suffers from malnutrition.

The phrase “extended immobilization”, as known in the art, describes a state in which a limb or the body is kept inactive for prolonged periods, often resulting in muscle atrophy. According to some of these embodiments, the subject suffers from extended immobilization.

The term “trauma”, as known in the art, describes physical injury that may result in disuse or direct damage to a muscle tissue. According to some of these embodiments, the subject suffers from such an injury.

The term “alcoholism”, as known in the art, describes chronic alcohol use disorder, which may lead to nutritional deficiencies and muscle wasting. According to some of these embodiments, the subject is a chronic alcohol user.

The phrase “cancer treatment”, as known in the art, describes therapeutic interventions for cancer (e.g., chemotherapy, radiation) that can lead to cachexia or muscle loss. According to some of these embodiments, the subject suffers from cancer or is treated with anti-cancer preventing therapy (e.g., to avoid recurrency).

The term “hyperthyroidism”, as known in the art, describes excessive thyroid hormone levels, which may lead to increased metabolism and muscle wasting. According to some of these embodiments, the subject suffers from hyperthyroidism.

The phrase “heart failure”, as known in the art, describes a condition where the heart cannot pump sufficient blood, often associated with cachexia and muscle loss. Non-limiting examples of heart failure conditions include systolic heart failure, diastolic heart failure, congestive heart failure, left-sided heart failure, and right-sided heart failure. According to some of these embodiments, the subject suffers or suffered from heart failure as described herein. According to some of these embodiments, the subject suffered from heart failure and is administered with medications to prevent heart failure recurrence.

The phrase “liver diseases”, as known in the art, describes disorders affecting liver function, which may disrupt metabolism and contribute to muscle wasting. Non-limiting examples of liver diseases include cirrhosis, hepatitis (e.g., hepatitis B and C), non-alcoholic fatty liver disease (NAFLD), alcoholic liver disease, and hepatocellular carcinoma. According to some of these embodiments, the subject suffers from a liver disease as described herein.

The phrase “kidney diseases”, as known in the art, describes chronic or acute impairments in kidney function, often leading to uremic cachexia and muscle loss. Non-limiting examples of kidney diseases include chronic kidney disease (CKD), acute kidney injury (AKI), end-stage renal disease (ESRD), glomerulonephritis, and diabetic nephropathy. According to some of these embodiments, the subject suffers from a kidney disease, as described herein.

The term “diabetes”, as known in the art, describes a metabolic disorder characterized by hyperglycemia, often associated with altered protein metabolism and muscle atrophy. Non-limiting types of diabetes include type 1 diabetes (insulin-dependent diabetes mellitus), type 2 diabetes (non-insulin-dependent diabetes mellitus), gestational diabetes, latent autoimmune diabetes in adults (LADA), maturity-onset diabetes of the young (MODY), and secondary diabetes (resulting from conditions such as pancreatitis, endocrine disorders, or medication use (e.g., corticosteroids)). According to some of these embodiments, the subject is a diabetic subject.

The term “osteoarthritis”, as known in the art, describes degenerative joint disease that may lead to decreased mobility and disuse atrophy. According to some of these embodiments, the subject suffers from osteoarthritis or osteopenia.

The phrase “nutritional atrophy”, as known in the art, describes muscle wasting due to inadequate dietary intake of essential nutrients.

The phrase “severe burns”, as known in the art, describes traumatic injuries causing systemic hypermetabolism and muscle catabolism

The phrase “malabsorption syndromes”, as known in the art, describes conditions impairing nutrient absorption in the gastrointestinal tract, leading to muscle loss.

The phrase “anorexia nervosa”, as known in the art, describes an eating disorder characterized by self-induced starvation, leading to severe muscle and fat loss.

According to some embodiments of any of the embodiments described herein, the muscle atrophy is not associated with a neurodegenerative disease or disorder, a genetic disease or disorder, and/or with motor neuron-related conditions.

According to some embodiments of any of the embodiments described herein, the muscle atrophy is not associated with a genetic disease or disorder. The phrase “genetic disease or disorder” in the context of the present embodiments describes a pathological condition that arises from a heritable or de novo mutation, deletion, duplication, or chromosomal rearrangement affecting one or more genes, and that contributes to the etiology or phenotype of the disorder. Non-limiting examples of genetic diseases or disorders include Duchenne muscular dystrophy, Becker muscular dystrophy, limb-girdle muscular dystrophy, congenital muscular dystrophy, facioscapulohumeral muscular dystrophy, spinal muscular atrophy (SMA), and Charcot-Marie-Tooth disease.

According to some embodiments of any of the embodiments described herein, the muscle atrophy is not associated with a neurodegenerative disease or disorder. By “neurodegenerative disorder” in the context of the present embodiments it is meant any disease or disorder caused by or associated with the deterioration of cells or tissues of the nervous system. Exemplary neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis.

According to some embodiments of any of the embodiments described herein, the muscle atrophy is not associated with motor neurons. The phrase “motor neurons” describes neurons that originate in the spinal cord or brainstem and project to muscles to control voluntary movement. As known in the art, the survival of motor neurons may affect a number of neurodegenerative disorders. Non-limiting such neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis. Motor neuron diseases (MNDs) are a group of neurodegenerative disorders that selectively target motor neurons, which are the nerve cells responsible for controlling voluntary muscle activities such as speaking, walking, breathing, swallowing, and general body movement. While these motor neuron diseases typically present distinct variations in their origins and causative factors, they share a common outcome for patients: profound muscle weakness. The classification of these disorders primarily depends on the principal location of motor neuron degeneration. As used herein, the phrase “motor neuron degeneration” means a condition of deterioration of motor neurons, wherein the neurons die or change to a lower or less functionally-active form. Non-limiting examples of MNDs include amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome. Other systemic conditions such as cancer, congestive heart failure, chronic obstructive pulmonary disease (COPD), AIDS, liver disease, renal failure, and cardiac failure may affect neuromuscular function secondarily, although they are not primarily classified as motor neuron disorders.

According to some embodiments of any of the embodiments described herein, the muscle atrophy is not associated with downregulation of activity and/or expression of a Survival of Motor Neuron protein and/or is not treatable by upregulating a Survival of Motor Neuron protein. The phrase “Survival of Motor Neuron protein” (SMN protein), as used herein describes a protein essential for the maintenance and function of motor neurons, the deficiency of which is implicated in genetic motor neuron disorders such as spinal muscular atrophy (SMA).

According to some embodiments of any of the embodiments described herein, the muscle atrophy is not associated with a neurodegenerative or genetic disorder, or with motor neuron-related conditions. Non-limiting examples of excluded disorders include spinal muscular atrophy (SMA), amyotrophic lateral sclerosis (ALS), primary lateral sclerosis (PLS), progressive muscular atrophy (PMA), progressive bulbar palsy (PBP), post-polio syndrome, multiple system atrophy, and spinal cord injury.

According to some embodiments of any of the embodiments described herein, the muscle atrophy is not associated with a motor neuron disease or with motor neuron degeneration, that is, a condition of deterioration of motor neurons, wherein the neurons die or change to a lower or less functionally-active form.

According to some embodiments of any of the embodiments described herein, the muscle atrophy is not associated with Spinal Muscular Atrophy (SMA).

Spinal Muscular Atrophy (SMA) refers to a number of different disorders, all having in common a genetic cause and the manifestation of weakness due to loss of the motor neurons of the spinal cord and brainstem. The most common form of SMA is caused by mutation of the SMN gene. Exemplary conditions include Werdnig-Hoffmann disease, scoliosis (curvature of the spine) and other skeletal abnormalities. Any of the compounds described herein can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound accountable for the biological effect, herein a GSK-3 inhibitor (e.g., GSK-3β inhibitor) as described herein in any of the respective embodiments and any combination thereof.

According to some embodiments of any of the embodiments described herein, the pharmaceutical composition comprises one or more pharmaceutically acceptable carriers, excipients, and binders.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Herein the term “binder” describes a substance that help the active and inactive ingredients of a formulation adhere to each other, promoting the integrity of the final dosage form during manufacturing, packaging, and handling. Non-limiting examples of pharmaceutically acceptable binders include starch, gelatin, cellulose derivatives (e.g., hydroxypropyl cellulose, hydroxypropyl methylcellulose), polyethylene glycol, polyvinylpyrrolidone (PVP), acacia, alginic acid, and sugars such as sucrose and glucose.

Techniques for formulation and administration of drugs may be found in “Remington's Pharmaceutical Sciences,” Mack Publishing Co., Easton, PA, latest edition, which is incorporated herein by reference.

A pharmaceutical composition as described herein can be formulated in accordance with a selected route of administration. Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

The phrase “muscle tissue”, as known in the art, describes a biological tissue composed primarily of contractile cells (muscle fibers) that generate force and facilitate movement, posture, and structural support. Muscle tissue includes skeletal muscle tissue, cardiac muscle tissue, and smooth muscle tissue as the major types, and muscle-like cells, which, although not traditionally classified as muscle tissue, exhibit contractile behavior and contribute to various physiological or pathological processes.

According to some embodiments, the muscle tissue is a skeletal muscle tissue. Skeletal muscles are voluntary, striated muscles attached to bones and are responsible for movement, posture, and joint stability. In the head and neck, muscles include the frontalis, occipitalis, orbicularis oculi, orbicularis oris, zygomaticus major and minor, buccinator, platysma, masseter, temporalis, medial and lateral pterygoids, sternocleidomastoideopezius, splenius capitis, scalenes, and levator scapulae. In the thorax and abdomen, key muscles are the pectoralis major and minor, serratus anterior, intercostals (external, internal, and innermost), rectus abdominis, external and internal obliques, and transversus abdominis. Back muscles include the erector spinae group (iliocostalis, longissimus, spinalis), latissimus dorsi, rhomboids, multifidus, and quadratus lumborum. Upper limb muscles encompass the deltoid, rotator cuff muscles (supraspinatus, infraspinatus, teres minor, subscapularis), biceps brachii, triceps brachii, brachialis, coracobrachialis, and numerous forearm and hand muscles such as the flexors and extensors of the wrist and fingers, pronator teres, supinator, lumbricals, interossei, and thenar and hypothenar muscles. Lower limb muscles include the gluteus maximus, medius, and minimus, iliopsoas, tensor fasciae latae, sartorius, quadriceps femoris group (rectus femoris, vastus lateralis, vastus medialis, vastus intermedius), hamstrings (biceps femoris, semitendinosus, semimembranosus), adductors (longus, brevis, magnus), gracilis, pectineus, as well as leg and foot muscles such as tibialis anterior and posterior, gastrocnemius, soleus, fibularis longus and brevis, extensor and flexor digitorum longus, flexor hallucis longus, and intrinsic muscles of the foot like abductor hallucis, abductor digiti minimi, and interossei.

According to some embodiments, the muscle tissue is of the upper limb, the lower limb, the leg and/or the foot, and optionally also of the head and/or neck.

Non-limiting examples of muscle-like cells include myofibroblasts (involved in wound contraction and fibrosis), myoepithelial cells (found in secretory glands such as mammary and salivary glands), and pericytes (associated with capillaries and microvessels, contributing to vascular stability and tone).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions, or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, non-irritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g., a GSK-3 inhibitor as described herein) effective to prevent, alleviate or ameliorate symptoms of a disorder (e.g., muscle atrophy as described herein) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

In some embodiments of any of the embodiments described herein, the GSK-3 inhibitor as described herein is used in combination with an additional therapeutically active agent.

In some embodiments of any of the embodiments described herein, the pharmaceutical composition further comprises one or more (additional) therapeutically active agent(s).

In some embodiments of any of the embodiments described herein, the method further comprises co-administering to the subject one or more (additional) therapeutically active agent(s)

According to some embodiments, the one or more (additional) therapeutically active agent is an additional GSK-3 inhibitor, as described herein in any of the respective embodiments and any combination thereof.

According to some embodiments, the one or more an (additional) therapeutically active agent is usable in treating or preventing muscle atrophy.

Non-limiting examples for therapeutically active agents usable in treating or preventing muscle atrophy include anti-catabolic agents (e.g., myostatin inhibitors such as follistatin or monoclonal antibodies against myostatin, activin receptor antagonists such as ACE-031); anabolic agents (e.g., testosterone, nandrolone, selective androgen receptor modulators such as ostarine (MK-2866), growth hormone secretagogues (GHS)); anti-inflammatory agents (e.g., corticosteroids such as dexamethasone, non-steroidal anti-inflammatory drugs (NSAIDs), cytokine inhibitors such as IL-6 or TNF-α inhibitors); antioxidants and mitochondrial function modulators (e.g., coenzyme Q10, L-carnitine, N-acetyl cysteine, mitochondrial-targeted peptides); β2-adrenergic agonists (e.g., clenbuterol, formoterol); neuromuscular activity modulators (e.g., cholinesterase inhibitors or agents that enhance neuromuscular transmission in neurogenic atrophy); antidiabetic agents (e.g., metformin and thiazolidinediones such as pioglitazone); nutritional supplements (e.g., leucine, HMB (β-hydroxy-β-methylbutyrate), creatine, and omega-3 fatty acids); and other small molecules or peptides (e.g., proteasome inhibitors such as MG-132, and agents that modulate autophagy or lysosomal pathways such as rapamycin, trehalose, spermidine).

According to some embodiments, the one or more (additional) therapeutically active agent is an agent or a therapy that causes or induces muscle atrophy, for example, anti-cancer therapy, and the GSK-3 inhibitor is co-administered or co-formulated with such an agent so as to attenuate the adverse side effect caused thereby.

Exemplary agents (medications) that induce or contribute to muscle atrophy or cachexia, either through direct catabolic effects on muscle tissue or via systemic metabolic disturbances, include, without limitation, glucocorticoids such as prednisone, dexamethasone, and hydrocortisone; chemotherapeutic agents, such as, for example, cisplatin, doxorubicin, paclitaxel, and methotrexate, and any other agent that causes cancer-associated cachexia; Immunosuppressants such as cyclosporine, sirolimus, and tacrolimus; antiretroviral drugs used in HIV therapy, such as zidovudine and stavudine; statins such as simvastatin and atorvastatin;

biologic agents targeting tumor necrosis factor-alpha, such as infliximab and etanercept; Loop diuretics like furosemide; Antiepileptic drugs such as phenytoin and valproic acid; heart failure medications such as beta-blockers and ACE inhibitors; and androgen-suppressing therapies including flutamide and leuprolide.

According to some embodiments of any of the embodiments described herein, the pharmaceutical composition does not comprise a nutrient.

According to some embodiments of any of the embodiments described herein, the pharmaceutical composition does not comprise one or more of glycine, mannitol, butyric acid, pyruvic acid, myoinositol, uridine, adenosine, 3-hydroxybutyric acid, alpha-ketoglutarate, Met-Trp, D-malic acid, sedoheptulosan, D-fructose, D-galactose, Leu-Asp, D-raffinose, D-lactitol, D,L-beta-hydroxy-butyric acid, gamma-hydroxy-butyric acid, Met-Pro, Meso-tartaric acid, N-acetyl-neuraminic acid, chondroitin-6 sulfate, L-rhamnose, meso-erythritol, pectin, Met-Thr, D-melezitose, succinamic acid, D-melibiose, propylene glycol, 2,3-butanediol, stachyose, beta-methyl -D-galactoside, palatinose, thymidine, methyl pyruvate, D,L-alpha-glycerol-phosphate, D-glucuronic acid, xylitol, inosine, methyl D-lactate, Leu-His, D-arabinose, mannan, D-fucose, succinic acid, alpha-methyl-D-galactoside, alpha-hydroxy-butyric acid, L-arabinose, hexanoic acid, alpha-methyl-D-mannoside, ethanolamine, N-acetyl-beta-D-mannosamine, D-fructose-6-phosphate, L-fucose, glycerol, beta-methyl-D-xylopyranoside, Met-Lys, adonitol, alpha-keto-butyric acid, gamma-amino-N-butyric acid, sucrose, acetic acid, Met-Met, Lys-Phe, 3-O-methyl-D-glucose, D-sorbitol, Met-Leu, D,L-lactic acid, lactulose, propionic acid, L-sorbose, maltitol, L-malic acid, L-glucose, alpha-D-lactose, alpha-methyl-D-glucoside, Leu-Gly, D-turanose, acetoacetic acid, Lys-Ser, Leu-Glu, alpha-cyclodextrin, Met-Tyr, L-histidine, 3-hydroxy-2-butanone, Ile-Ile, D-cellobiose, D-salicin, Met-Val, tricarballylic acid, His-Trp, and any salts thereof (e.g., pharmaceutically acceptable salts, e.g., sodium salt).

According to some embodiments of any of the embodiments described herein, the pharmaceutical composition does not comprise one or more of adenosine, myoinositol, glycine, alpha-ketoglutarate, sodium butyrate and mannitol.

As used herein throughout, the term “subject” includes mammals, preferably human beings at any age which suffer, or is at risk of developing muscle atrophy, for example, who are afflicted by a condition associated with muscle atrophy as described herein. According to some embodiments of any of the embodiments described herein, the treatment is of a subject suffering from, afflicted by, or who is at risk of being afflicted by, muscle atrophy and/or any of the medical conditions described herein.

According to some embodiments of any of the embodiments described herein, the subject is a healthy subject who is in need of, or is willing to increase muscle mass and/or strength or prevent loss of muscle mass and/or strength.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression (e.g., preventing or reducing progression) of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

Herein, the phrase “linking group” describes a group (e.g., a substituent) that is attached to two or more moieties in the compound; whereas the phrase “end group” describes a group (e.g., a substituent) that is attached to a single moiety in the compound via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, as defined herein, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ group or a —N+R′R″R″′ group, wherein R′, R″ and R″′ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R″′ are hydrogen or alkyl comprising 1 to 4 carbon atoms. Optionally, R′ and R″ (and R″′, if present) are hydrogen. When substituted, the carbon atom of an R′, R″ or R″′ hydrocarbon moiety which is bound to the nitrogen atom of the amine is not substituted by oxo (unless explicitly indicated otherwise), such that R′, R″ and R″′ are not (for example) carbonyl, C-carboxy or amide, as these groups are defined herein.

An “azide” group refers to a —N═N⁺═N⁻ end group.

An “alkoxy” group refers to any of an —O-alkyl, —O-alkenyl, —O-alkynyl, —O-cycloalkyl, and —O-heteroalicyclic end group, as defined herein, or to any of an —O-alkylene, —O-cycloalkyl- and —O-heteroalicyclic-linking group, as defined herein.

An “aryloxy” group refers to both an —O-aryl and an —O-heteroaryl group, as defined herein, or to an —O-arylene.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic end group, as defined herein, or to any of an —S-alkylene-, —S-cycloalkyl- and —S-heteroalicyclic-linking group, as defined herein.

A “thioaryloxy” group refers to both an —S-aryl and an —S-heteroaryl group, as defined herein, or to an —S-arylene.

A “carbonyl” or “acyl” group refers to a —C(═O)—R′ end group, where R′ is defined as hereinabove, or to a —C(═O)— linking group.

A “thiocarbonyl” group refers to a —C(═S)—R′ end group, where R′ is as defined herein, or to a —C(═S)— linking group.

A “carboxy”, “carboxyl”, “carboxylic” or “carboxylate” group refers to both “C-carboxy” and “O-carboxy” end groups, as defined herein, as well as to a carboxy linking group, as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxy linking group” refers to a —C(═O)—O— linking group.

An “oxo” group refers to a ═O end group.

An “imine” group refers to a ═N—R′ end group, where R′ is as defined herein, or to an ═N-linking group.

An “oxime” group refers to a ═N—OH end group.

A “hydrazone” group refers to a ═N—NR′R″ end group, where each of R′ and R″ is as defined herein, or to a ═N—NR′— linking group where R′ is as defined herein.

A “halo” group refers to fluorine, chlorine, bromine or iodine.

A “sulfinyl” group refers to an —S(═O)—R′ end group, where R′ is as defined herein, or to an —S(═O)— linking group.

A “sulfonyl” group refers to an —S(═O)₂—R′ end group, where R′ is as defined herein, or to an —S(═O)₂— linking group.

A “sulfonate” group refers to an —S(═O)₂—O—R′ end group, where R′ is as defined herein, or to an —S(═O)₂—O— linking group.

A “sulfate” group refers to an —O—S(═O)₂—O—R′ end group, where R′ is as defined as herein, or to an —O—S(═O)₂—O— linking group.

A “sulfonamide” or “sulfonamido” group encompasses both S-sulfonamido and N-sulfonamido end groups, as defined herein, as well as a sulfonamide linking group, as defined herein.

An “S-sulfonamido” group refers to a —S(═O)₂—NR′R″ end group, with each of R′ and R″ as defined herein.

An “N-sulfonamido” group refers to an R′S(═O)₂—NR″— end group, where each of R′ and R″ is as defined herein.

A “sulfonamide linking group” refers to a —S(═O)₂—NR′— linking group, where R′ is as defined herein.

A “carbamyl” group encompasses both O-carbamyl and N-carbamyl end groups, as defined herein, as well as a carbamyl linking group, as defined herein.

An “O-carbamyl” group refers to an —OC(═O)—NR′R″ end group, where each of R′ and R″ is as defined herein.

An “N-carbamyl” group refers to an R′OC(═O)—NR″— end group, where each of R′ and R″ is as defined herein.

A “carbamyl linking group” refers to a —OC(═O)—NR′— linking group, where R′ is as defined herein.

A “thiocarbamyl” group encompasses O-thiocarbamyl, S-thiocarbamyl and N-thiocarbamyl end groups, as defined herein, as well as a thiocarbamyl linking group, as defined herein.

An “O-thiocarbamyl” group refers to an —OC(═S)—NR′R″ end group, where each of R′ and R″ is as defined herein.

An “N-thiocarbamyl” group refers to an R′OC(═S)NR″— end group, where each of R′ and R″ is as defined herein.

An “S-thiocarbamyl” group refers to an —SC(═O)—NR′R″ end group, where each of R′ and R″ is as defined herein.

A “thiocarbamyl linking group” refers to a —OC(═S)—NR′— or —SC(═O)—NR′— linking group, where R′ is as defined herein.

An “amide” or “amido” group encompasses C-amido and N-amido end groups, as defined herein, as well as an amide linking group, as defined herein.

A “C-amido” group refers to a —C(═O)—NR′R″ end group, where each of R′ and R″ is as defined herein.

An “N-amido” group refers to an R′C(═O)—NR″— end group, where each of R′ and R″ is as defined herein.

An “amide linking group” refers to a —C(═O)—NR′— linking group, where R′ is as defined herein.

A “urea group” refers to an —N(R′)—C(═O)—NR″R′″ end group, where each of R′, R″ and R″ is as defined herein, or an —N(R′)—C(═O)—NR″— linking group, where each of R′ and R″ is as defined herein.

A “thiourea group” refers to an —N(R′)—C(═S)—NR″R″′ end group, where each of R′, R″ and R″ is as defined herein, or an —N(R′)—C(═S)—NR″— linking group, where each of R′ and R″ is as defined herein.

A “nitro” group refers to an —NO₂ group.

A “cyano” group refers to a —C═N group.

The term “phosphonyl” or “phosphonate” describes a —P(═O)(OR′)(OR″) group, with R′ and R″ as defined herein, or a —P(═O)(OR′)—O— linking group, with R′ as defined herein.

The term “phosphate” describes an —O—P(—O)(OR′)(OR″) end group, with each of R′ and R″ as defined herein, or an —O—P(—O)(OR′)—O— linking group, with R′ as defined herein.

The term “phosphinyl” describes a —PR′R″ end group, with each of R′ and R″ as defined herein, or a —PR′— linking group, with R′ as defined herein.

The term “hydrazine” describes a —NR′—NR″R″′ end group, where R′, R″, and R″′ are as defined herein, or to a —NR′—NR″— linking group, where R′ and R″ are as defined herein.

As used herein, the term “hydrazide” describes a —C(═O)—NR′—NR″R″′ end group, where R′, R″ and R′″ are as defined herein, or to a —C(═O)—NR′—NR″— linking group, where R′ and R″ are as defined herein.

As used herein, the term “thiohydrazide” describes a —C(═S)—NR′—NR″R″′ end group, where R′, R″ and R′″ are as defined herein, or to a —C(═S)—NR′—NR″— linking group, where R′ and R″ are as defined herein.

A “guanidinyl” group refers to an —RaNC(═NRd)—NRbRc end group, where each of Ra, Rb, Rc and Rd can be as defined herein for R′ and R″, or to an —R′NC(═NR″)—NR″′— linking group, where R′, R″ and R′″ are as defined herein.

A “guanyl” or “guanine” group refers to an R″′R″NC(═NR′)— end group, where R′, R″ and R′″ are as defined herein, or to a —R″NC(═NR′)-linking group, where R′ and R″ are as defined herein.

For any of the embodiments described herein, the compound as described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an(S) configuration and any combination, and compounds according to some embodiments of the present invention, can have any their chiral centers exhibit an (R) or an(S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

CHIR98014 and CHIT99021 (see, FIG. 2), commercially available ATP-competitive GSK-3 inhibitors (CAS Nos. 252935-94-7 and 252917-06-9, respectively) were obtained from Sigma Aldrich.

L803-mts (SEQ ID NO:2) was obtained from Calbiochem.

Tideglusib (CAS No. 865854 May 3) (See, FIG. 2) was obtained from Sigma Aldrich.

shLacZ plasmid was generated using BLOCK-It INVITROGEN K4936-02 (Thermo Fischer Scientific).

GSK-3-β-DN plasmid was obtained from Addgene.

All animal experiments were consistent with Israel Council on Animal Experiments guidelines and the Institutional Regulations of Animal Care and Use. Specialized personnel provided mice care in the institutional animal facility. In fasting (2 d) experiments, food was removed from cages on the fifth day after electroporation.

### Example 1

### Design

Some of the present inventors previously studied the role of desmin depolymerization in overall protein degradation during atrophy. It was found that phosphorylation by protein kinase GSK3-β is essential for the depolymerization of desmin filaments by calpain-1, leading to myofibril destruction in muscle atrophy (Aweida et al., J Cell Biol. 2018 Oct 1;217(10):3698-3714).

To further study the effect of GSK3-β inhibition on muscle wasting, the muscle weight loss (%) was evaluated in mice transfected with GSK-3-β-dominant negative (DN) plasmid, having reduced GSK-3-β function.

Mice (25-29 grams) were first anaesthetized, after which buprenorphine was administered at a dose of 0.05 mg/kg body weight, following lab protocols for pain management. The mice's legs were then secured to a surgery board to ensure stability during the procedure, after which their lower leg was shaved and sterilized with alcohol pads and iodine. The surgical phase began with a longitudinal incision over the tibialis anterior (TA) muscle, approximately 1 cm in length, extending from just below the knee to slightly above the ankle, while avoiding the larger vein on the lateral surface of the ankle. Optionally, the fascia surrounding the TA was opened to prevent compression of the muscle, which is crucial for successful transfection. Curved forceps were gently placed under the TA muscle to facilitate the positioning of a plate electrode beneath the TA and perpendicular to it, followed by the removal of the forceps. A plasmid mixture was then injected using a 100 μL Hamilton syringe with a 0.5 inch, beveled, 30 or 31G needle, comprising 20 μg plasmid in 4 microliter 0.9% NaCl, made up to 40 μL with MiliQ water. Of the plasmids mixtures injected, fed mice were injected with the shLacz plasmid to serve as a control, and the fasted mice were injected with either the shLacz plasmid or GSK-3-β-dominant negative (DN) plasmid, at the TA muscles. The injection of the anesthesia was performed intraperitoneally, angling the needle towards the knee to align with the muscle fibers, ensuring even distribution even if repositioning is necessary due to uneven swelling. Electroporation was applied, using an ECM 830 BTX Electroporator, with plate electrodes positioned on and parallel to the TA without contact. V of electric pulse was applied for mice weighing 20-40 grams using the two electrodes (five pulses, 200-ms intervals), adjusting voltage as needed for mice outside this weight range to prevent muscle damage. Following the electroporation, the skin incision was closed with 3-4 sutures to complete the procedure. The muscle weight loss (%) was then evaluated in comparison to the fed mice transfected with shLacz.

FIG. 1 presents a bar graph depicting the muscle weight (%) of muscle isolated from mice, expressing shLacz (fed) and shLacz or GSK-3-β-DN fasting for 2 days, as a percentage of the shLacz fed control.

As can be seen in FIG. 1, injecting mouse muscle with GSK-3-β-DN blocked muscle atrophy (*p<0.05 vs. shLacz in atrophy by unpaired Student's t-test) in comparison to the shLacz control, confirming that inhibition of GSK-3-β suppresses muscle atrophy.

### Example 2

### Effect of CHIR98014 on Muscle Wasting in Mice

The present inventors turned to studying the effect of GSK3-β inhibitors on muscle wasting. Two exemplary GSK3-β inhibitors, CHIR-98014 (ATP-competitive inhibitor; See, FIG. 2) and L803-mts (SEQ ID NO:2) (substrate-competitive inhibitor), were studied for their effect on muscle wasting in mice.

Mice were divided into two groups: those subjected to fasting to induce muscle atrophy, and those that were fed and used as fed control. Mice of the fasting group were injected intraperitoneally with either CHIR98014 or L803mts to examine their effect on muscle wasting. Control groups (Fed, fasting) were injected with vehicle (3% DMSO in saline). Each mouse (27-30 grams body weight) was injected with 0.3 mg L803mts or 0.5 mg CHIR98014, twice: on the first and then on the second day of fasting. Whole Tibilis anterior (TA) muscles were then collected and the weight loss in these samples over 2 days was measured.

FIG. 3 presents a bar graph depicting the mouse TA muscle weight in fed control mice, fasting control mice, fasting mice injected with CHIR98014 and fasting mice injected with L803-mts (SEQ ID NO:2).

As can be seen in FIG. 3, while control (vehicle-treated) fasting mice exhibited a reduction in muscle weight, indicative of muscle atrophy, an attenuation in muscle weight was observed in fasting mice injected with L803-mts (SEQ ID NO:2). However, mice injected with CHIR98014 exhibited no reduction in muscle weight compared to control, indicating no muscle atrophy, and superior performance over L803-mts (SEQ ID NO:2).

In order to further study the effect of other GSK-3 inhibitors in comparison with GSK-3 inhibitors as disclosed herein, fasting (atrophy-induced) mice were treated with the GSK-3β inhibitor Tideglusib in comparison with fed mice (control) and fasting (atrophy-induced) mice, and mice.

Tideglusib (4.35 mg/ml) was mixed with 26% PEG400 (v/v), 15% Cremophor EL (v/v), and 59% ddH₂O (v/v), to a total volume of 2.3 ml. Each mouse received 200 microliter (μL) (0.87 mg Tideglusib) of the solution on the first day of fasting, and 150 μL (0.65 mg Tideglusib) on the second day.

FIGS. 4A and 4B present bar graphs illustrating body weight and TA muscle-to-body weight ratios, respectively. The data indicate that Tideglusib had a minimal effect on preventing muscle atrophy under these experimental conditions.

Then, the efficacy of CHIR99021, another exemplary ATP-competitive GSK3β inhibitor, was evaluated. To this, fasting (atrophy-induced) mice were injected intraperitoneally with 16 mg/kg CHIR99021 (200 μL per mouse) on day 1 and on day 2 of fasting. At the end of the experiment, mice were sacrificed, and their tibialis anterior (TA) muscles were dissected and weighed, in comparison with fed mice and untreated fasting (atrophy-induced) mice.

The results (data not shown) demonstrated that fasting mice experienced approximately a 10% reduction in TA muscle weight compared to fed controls. Treatment with CHIR99021 did not mitigate this muscle loss, indicating limited efficacy in preventing muscle atrophy in this model.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for treating muscle atrophy in a subject in need thereof, the method comprising administering the subject with a therapeutically effective amount of a compound represented by Formula I: 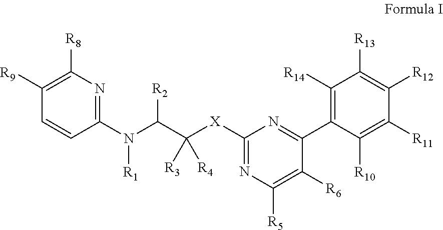
wherein:
X is O, NR₁₅, or CR₁₅R₁₆;
R₁, R₂, R₃ and R₄ are independently selected from hydrogen, hydroxy, thiol, alkyl, cycloalkyl, alkoxy, amine, aryl, alkaryl, heteroaryl, and heteroalicyclic;
R₅ is selected from hydrogen, halo, alkyl, cycloalkyl, alkoxy, thioalkoxy, amine, aryl, alkaryl, heteroaryl, heteroalicyclic, amide, thioamide and sulfonamide;
R₆ is selected from hydrogen, hydroxy, thiol, halo, carboxy, nitro, amine, amide, thioamide, cyano, alkyl, cycloalkyl, aryl, alkaryl, heteroaryl, heteroalicyclic, alkoxy, thioalkoxy, formyl, amide, sulfonyl, sulfonamide, and guanidinyl;
R₈ and R₉ are independently selected from hydrogen, nitro, amine, cyano, halo, thioamide, amide, oxime, guanidinyl, sulfonamide, carboxy, formyl, alkyl, cycloalkyl, aryl, alkaryl, heteroaryl and heteroalicyclic;
R₁₀, R₁₁, R₁₂, R₁₃ and R₁₄ are independently selected from hydrogen, nitro, amine, cyano, halo, thioamide, carboxy, hydroxy, thiol, amide, thioamide, alkyl, cycloalkyl, aryl, alkaryl, heteroaryl and heteroalicyclic; and
R₁₅ and R₁₆ are each independently selected from hydrogen, hydroxy, thiol, alkyl, cycloalkyl, alkoxy, amine, aryl, alkaryl, heteroaryl, and heteroalicyclic, or a pharmaceutically acceptable salts thereof.

2. The method of claim 1, wherein X is NR₁₅.

3. The method of claim 2, wherein R₁₅ is hydrogen.

4. The method of claim 1, wherein at least one of R₈ and R₉ is selected from of nitro, amine, cyano, alkyl and alkoxy.

5. The method of claim 1, wherein at least one of R₈ and R₉ is selected from nitro, amine, alkyl and alkoxy.

6. The method of claim 1, wherein R₈ is an amine.

7. The method of claim 1, wherein R₉ is a nitro.

8. The method of claim 1, wherein each of R₁, R₂, R₃ and R₄ is hydrogen.

9. The method of claim 1, wherein R₅ is selected from hydrogen, aryl and heteroaryl.

10. The method of claim 1, wherein R₅ is hydrogen.

11. The method of claim 1, wherein at least one of R₁₀, R₁₁, R₁₂, R₁₃ and R₁₄ is other than hydrogen and is selected from halo, alkyl, hydroxy, alkoxy, amide and cyano.

12. The method of claim 1, wherein R₁₀, R₁₁ and R₁₃ are each hydrogen and at least one or both of R₁₂ and R₁₄ are each independently selected from halo, alkyl, hydroxy, alkoxy, amide and cyan.

13. The method of claim 8, wherein R₁₂ and R₁₄ are each independently halo (e.g., chloro).

14. The method of claim 1, wherein R₆ is a heteroaryl.

15. The method of claim 11, wherein R₆ is selected from pyridyl, pyrimidinyl, pyrrolindinyl, thiazolyl, indolyl, imidazolyl, oxadiazolyl, tetrazolyl, pyrazinyl, triazolyl, thienyl, furanyl, quinolinyl, pyrrolylpyridyl, benzothiazolyl, benzopyridyl, benzotriazolyl, and benzimidazolyl, each being optionally substituted.

16. The method of claim 1, wherein R₆ is an imidazole.

17. The method of claim 16, wherein said compound is represented by Formula III: 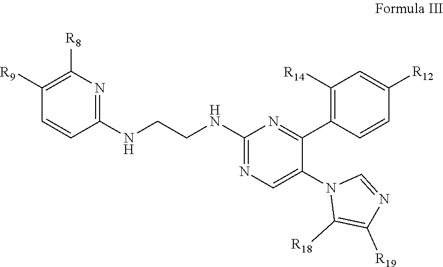
wherein:
R₁₂ and R₁₄ are each independently halo (e.g., chloro);
R₁₈ and R₁₉ are each independently selected from hydrogen, alkyl, cycloalkyl, aryl, and is preferably selected from hydrogen and alkyl; and
R₈ and R₉ are each independently selected from hydrogen, amine, cyano, nitro and alkyl.

18. The method of claim 1, wherein the compound is:

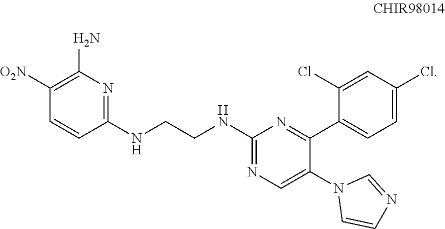 [SMILES: Nc1nc(NCCNc2ncc(-n3ccnc3)c(-c3ccc(Cl)cc3Cl)n2)ccc1[N+](=O)[O-]]

19. The method claim 1, wherein the compound forms a part of a pharmaceutical composition which further comprises a pharmaceutically acceptable carrier.

20. The method of claim 1, wherein the muscle atrophy is associated with at least one of cachexia, sedentary lifestyle, sarcopenia, malnutrition, disuse atrophy, neurogenic atrophy, amyotrophic lateral sclerosis (ALS), Duchenne muscular dystrophy, myotonic dystrophy, Becker muscular dystrophy, facioscapulohumeral muscular dystrophy, limb-girdle muscular dystrophy, Charcot-Marie-Tooth disease, peripheral neuropathy, corticosteroid therapy, Emery-Dreifuss muscular dystrophy, Distal muscular dystrophy, Oculopharyngeal muscular dystrophy, Congenital muscular dystrophy, neuromuscular diseases, extended immobilization, trauma, alcoholism, cancer treatment, hyperthyroidism, heart failure, liver diseases, kidney diseases, diabetes, osteoarthritis, Cushing's syndrome, nutritional atrophy, severe burns, malabsorption syndromes, anorexia nervosa and ischemic atrophy, anorexia nervosa, rheumatoid arthritis and surgery.

21. The method of claim 1, further comprising administering to the subject an additional therapeutically agent, said additional therapeutically active agent being selected from an agent usable in treating muscle atrophy, an additional GSK-3 inhibitor, and an agent that induces muscle atrophy.


SEQUENCE LISTING (2 sequences)

SEQ ID NO: 1 - protein, 420 aa, unidentified
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFFNTTQELSSNPPLATILIPPHARIQAAASTPTNATAASDANTGDRGQTNNAASASASNST

SEQ ID NO: 2 - protein, 12 aa, synthetic construct
  MOD_RES 1: note = N-myristoylated Gly
  MOD_RES 11: note = phosphorylated Ser
  MOD_RES 12: note = C-terminal amide
GKEAPPAPPQSP